(19) United States
(12) Patent Application Publication
(10) Pub. No.: US 2026/0004936 A1
(43) Pub. Date: Jan. 1, 2026

(54) OPTIMIZING CARE MANAGEMENT INTERVENTIONS USING PREDICTIVE MODELS

(71) Applicant: Elevance Health, Inc., Indianapolis, IN (US)

(72) Inventors: Ayush Mathur, Novi, MI (US); Kimberly A. Cole, Virginia Beach, VA (US); Geeta Sehgal, Sparta, NJ (US); Kimberly Cowart, Columbus, GA (US); Melissa Trownsell, Lafayette, IN (US); Omar Latif, Irvine, CA (US); Eugene Hsu, Burlingame, CA (US)

(21) Appl. No.: 19/253,100

(22) Filed: Jun. 27, 2025

Related U.S. Application Data
(60) Provisional application No. 63/665,430, filed on Jun. 28, 2024.

(51) Int. Cl. G16H 50/30; G16H 50/70

(52) U.S. Cl. CPC G16H 50/30; G16H 50/70

(57) ABSTRACT

A first risk score for each member in a population is generated using a first predictive model. A second risk score is generated for each member in the population using a second predictive model. The population is stratified into a plurality of risk groups based on: (i) predefined manageable medical conditions; (ii) the first risk score; and (iii) the second risk score. Based on the plurality of risk groups, the population is segmented into a high-risk group and a low-risk group. A panel of care management associates of a plurality of care management associates is assigned to each member in the member population based on whether the member belongs to the high-risk group or the low-risk group.

### CROSS-REFERENCE TO RELATED APPLICATIONS

This application claims priority to and the benefit of U.S. Provisional Application No. 63/665,430, filed Jun. 28, 2024, the entirety of which is incorporated herein by reference.

### TECHNICAL FIELD

This application generally relates to predictive modeling, and more particularly, to predictive modeling for improving care management services.

### BACKGROUND

Care management in healthcare refers to a collaborative and patient-centered approach to delivering healthcare services. It involves coordinating and organizing care activities to ensure that individuals receive the right care at the right time, with a focus on improving patient outcomes and enhancing the overall healthcare experience. Care management is particularly important for individuals with complex or chronic health conditions who may require ongoing and coordinated care from multiple healthcare providers. The goal of care management is to improve the quality of care, enhance patient satisfaction, and reduce healthcare costs, for example, by preventing unnecessary hospitalizations and emergency room visits. It promotes a more integrated and holistic approach to healthcare, recognizing that effective care goes beyond individual medical treatments and encompasses the broader needs of the patient.

Not all patients with chronic conditions are good candidates for care management. The process of identifying patients in a population of patients at a particular point in time that will benefit the most from respective care services is challenging and inefficient. Cara management services are costly, and resources are limited. Identifying patients that are at high risk of poor health outcomes, patients that are most likely to be positively impacted by care management services, and patients that are willing to engage and follow treatment plans care is challenging.

### SUMMARY

Provision of care management services to patients has been inefficient and a costly process. Paneling processes are manual and suggested interventions are based on limited information. For example, inefficient care management interventions are triggered based on limited predetermined scenarios (e.g., when patients are discharged or specific claims are processed) and such interventions do not dynamically and automatically update based on patient needs, costs, available resources, and/or capacity of care management personnel. Accordingly, there is a need for computer systems with improved methods for identifying patients in a population of patients that are suitable candidates for care management services and for generating care intervention alerts.

The above inefficiencies and other problems associated with the provision of care management services are reduced or eliminated by the disclosed methods and systems. Such methods and systems identify and stratify a population of members or patients into key groups of risk based on overall complexity, clinical impactibility (e.g., likelihood of being impacted positively by care management services), and the risk of future events (e.g., risk of inpatient admission. Predictive models use a unique combination of data, such as demographic, previous interaction (e.g., interaction with care management staff), service authorization data which dictates medical necessity, cost, and utilization data (Rx, labs, ambulatory, etc.) to develop risk profiles of respective members in the member population.

In various embodiments, a system is disclosed. The system includes a non-transitory memory and a processor communicatively coupled to the non-transitory memory. The processor is configured to read a set of instructions to generate a first risk score for each member in a population using a first predictive model. The processor is further configured to read the set of instructions to generate a second risk score for each member in the population using a second predictive model different from the first predictive model. The processor is further configured to read the set of instructions to stratify the population into a plurality of risk groups based on: (i) predefined manageable medical conditions; (ii) the first risk score; and (iii) the second risk score. The processor is configured to read a set of instructions to, based on the plurality of risk groups, segment the population into a high-risk group and a low-risk group, including assigning members of a first subset of the plurality of risk groups to the high-risk group and assigning members of a second subset of the plurality of risk groups to the low-risk group. The processor is configured to read a set of instructions to assign a panel of one or more care management associates of a plurality of care management associates to each member in the member population based on whether the member belongs to the high-risk group or the low-risk group, wherein the plurality of care management associates are segmented into a high-risk group and a low-risk group.

In various embodiments, a computer-implemented method is disclosed. The computer-implemented method includes steps of generating a first risk score for each member in a population using a first predictive model. The computer-implemented method further includes the steps of generating a second risk score for each member in the population using a second predictive model different from the first predictive model. The computer-implemented method further includes the steps of stratifying the population into a plurality of risk groups based on: (i) predefined manageable medical conditions; (ii) the first risk score; and (iii) the second risk score. The computer-implemented method further includes the steps of, based on the plurality of risk groups, segmenting the population into a high-risk group and a low-risk group, including assigning members of a first subset of the plurality of risk groups to the high-risk group and assigning members of a second subset of the plurality of risk groups to the low-risk group. The computer-implemented method further includes the steps of assigning a panel of one or more care management associates of a plurality of care management associates to each member in the member population based on whether the member belongs to the high-risk group or the low-risk group, wherein the plurality of care management associates are segmented into a high-risk group and a low-risk group.

In various embodiments, a non-transitory computer readable medium having instructions stored thereon is disclosed. The instructions, when executed by at least one processor, cause at least one device to perform operations including generating a first risk score for each member in a population using a first predictive model. The device is further configured to perform operations including generating a second risk score for each member in the population using a second predictive model different from the first predictive model. The device is further configured to perform operations including stratifying the population into a plurality of risk groups based on: (i) predefined manageable medical conditions; (ii) the first risk score; and (iii) the second risk score. The device is further configured to perform operations including, based on the plurality of risk groups, segmenting the population into a high-risk group and a low-risk group, including assigning members of a first subset of the plurality of risk groups to the high-risk group and assigning members of a second subset of the plurality of risk groups to the low-risk group. The device is further configured to perform operations including assigning a panel of one or more care management associates of a plurality of care management associates to each member in the member population based on whether the member belongs to the high-risk group or the low-risk group, wherein the plurality of care management associates are segmented into a high-risk group and a low-risk group.

### BRIEF DESCRIPTION OF THE DRAWINGS

The features and advantages of the present invention will be more fully disclosed in, or rendered obvious by the following detailed description of the preferred embodiments, which are to be considered together with the accompanying drawings wherein like numbers refer to like parts and further wherein:

FIG. 1 illustrates a network environment configured to generate automated care management intervention, in accordance with some embodiments.

FIG. 2 illustrates a computer system configured to implement one or more processes, in accordance with some embodiments.

FIG. 3 is a flowchart illustrating a method for generating automated care management intervention, in accordance with some embodiments.

FIG. 4 is a block diagram illustrating various portions of a care management stratification engine, in accordance with some embodiments.

FIG. 5 is a block diagram illustrating various portions of a care management system, in accordance with some embodiments.

FIG. 6 illustrates an artificial neural network, in accordance with some embodiments;

FIG. 7 illustrates a tree-based artificial neural network, in accordance with some embodiments;

FIG. 8 illustrates a deep neural network (DNN), in accordance with some embodiments;

FIG. 9 is a flowchart illustrating a training method for generating a trained machine learning model, in accordance with some embodiments; and

FIG. 10 is a process flow illustrating various steps of the training method of FIG. 9, in accordance with some embodiments.

### DETAILED DESCRIPTION

This description of the exemplary embodiments is intended to be read in connection with the accompanying drawings, which are to be considered part of the entire written description. Terms concerning data connections, coupling and the like, such as “connected” and “interconnected,” and/or “in signal communication with” refer to a relationship wherein systems or elements are electrically connected (e.g., wired, wireless, etc.) to one another either directly or indirectly through intervening systems, unless expressly described otherwise. The term “operatively coupled” is such a coupling or connection that allows the pertinent structures to operate as intended by virtue of that relationship.

In the following, various embodiments are described with respect to the claimed systems as well as with respect to the claimed methods. Features, advantages, or alternative embodiments herein may be assigned to the other claimed objects and vice versa. In other words, claims for the systems may be improved with features described or claimed in the context of the methods. In this case, the functional features of the method are embodied by objective units of the systems. While the present disclosure is susceptible to various modifications and alternative forms, specific embodiments are shown by way of example in the drawings and will be described in detail herein. The objectives and advantages of the claimed subject matter will become more apparent from the following detailed description of these exemplary embodiments in connection with the accompanying drawings.

Furthermore, in the following, various embodiments are described with respect to methods and systems for automated care management intervention. In various embodiments, a first risk score and a second risk score are generated for each member in a population using a first predictive model and a second predictive model, respectively, that is different from the first predictive model. The population is stratified (e.g., group, segmented, or classified) into a plurality of risk groups based on: (i) predefined manageable medical conditions; (ii) the first risk score; and (iii) the second risk score. Based on the plurality of risk groups, the population is further segmented into a high-risk group and a low-risk group. For example, members of a first subset of the plurality of risk groups (e.g., groups 3, 4, and 5 out of 5) are assigned to the high-risk group and members of a second subset of the plurality of risk groups (e.g., groups 0, 1, and 2 out of 5) are assigned to the low-risk group. A panel of one or more care management associates of a plurality of care management associates is assigned to each member in the member population based on whether the member belongs to the high-risk group or the low-risk group, wherein the plurality of care management associates are segmented into a high-risk group and a low-risk group. In various embodiments, alerts are optionally automatically generated and communicated to respective panels of care management associates optionally using a predictive model. In some embodiments, based on assigned risk group and other factors, a predictive model generates respective alerts for the care management panels. An alerts interface may be generated including interface elements configured to display member profiles, including risk scores or risk group classifications, medical conditions, associated cost, and other relevant parameters, and elements of alerts or suggestions for a selected electronic communication intervention plan, and/or any other suitable information.

In some embodiments, systems, and methods for automated care management intervention includes one or more trained cost prediction models, illness severity prediction models, and/or alert generation models. The trained cost prediction models, illness severity prediction models, and/or alert generation models may include one or more models, such as including supervised machine learning, decision trees, ensemble learning, and/or gradient boosting, logistic regression, etc.

In general, a trained function mimics cognitive functions that humans associate with other human minds. In particular, by training based on training data the trained function is able to adapt to new circumstances and to detect and extrapolate patterns.

In general, parameters of a trained function may be adapted by means of training. In particular, a combination of supervised training, semi-supervised training, unsupervised training, reinforcement learning and/or active learning may be used. Furthermore, representation learning (an alternative term is “feature learning”) may be used. In particular, the parameters of the trained functions may be adapted iteratively by several steps of training.

In some embodiments, a trained function may include a neural network, a support vector machine, a decision tree, a Bayesian network, a clustering network, Qlearning, genetic algorithms and/or association rules, and/or any other suitable artificial intelligence architecture. In some embodiments, a neural network may be a deep neural network, a convolutional neural network, a convolutional deep neural network, etc. Furthermore, a neural network may be an adversarial network, a deep adversarial network, a generative adversarial network, etc.

In various embodiments, neural networks which are trained (e.g., configured or adapted) to generate medical cost risk score and illness severity risk score, are disclosed. A neural network trained to generate medical cost risk may be referred to as a trained cost prediction model and a neural network trained to generate illness severity risk score may be referred to as a trained illness severity prediction model. A trained cost prediction model and illness severity prediction model may be configured to receive a set of input data, including, but not limited to, demographics, clinical historical data, care management history (e.g., prior interactions with care management, prescribed and/or performed treatments, and/or utilized or unutilized care management services) performed lab tests and respective lab results, historical data regarding pharmacy use (e.g., fulfilled and/or unfulfilled prescriptions, history of chronic and ambulatory sensitive conditions, overall cost and utilization of care services and benefits, authorization requests, and others.

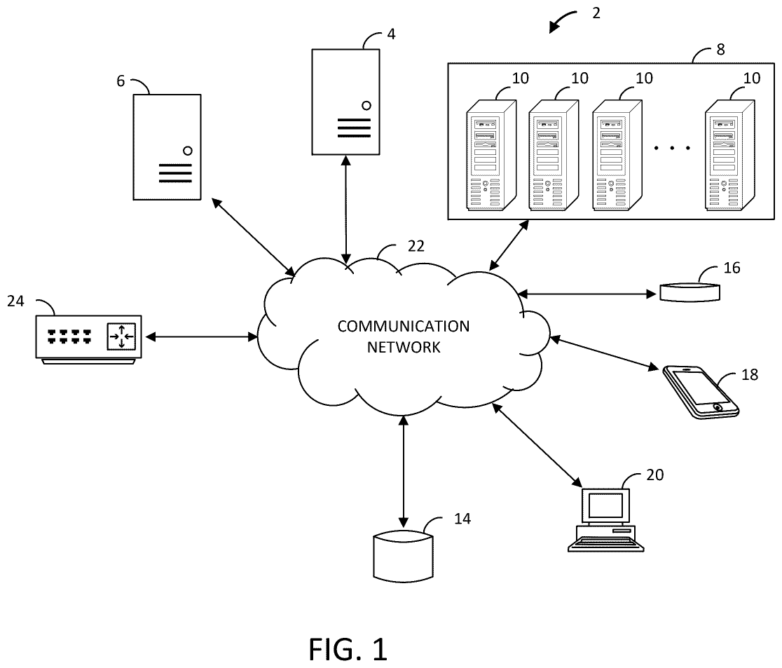

FIG. 1 illustrates a network environment 2 configured to provide automated care management intervention, in accordance with some embodiments. The network environment 2 includes a plurality of devices or systems configured to communicate over one or more network channels, illustrated as a network cloud 22. For example, in various embodiments, the network environment 2 may include, but is not limited to, a care management computing device 4, a web server 6, a cloud-based engine 8 including one or more processing devices 10, a database 14, and/or one or more user computing devices 16, 18, 20 operatively coupled over the network 22. The care management computing device 4, the web server 6, the processing device(s) 10, and/or the user computing devices 16, 18, 20 may each be a suitable computing device that includes any hardware or hardware and software combination for processing and handling information. For example, each computing device may include, but is not limited to, one or more processors, one or more field-programmable gate arrays (FPGAs), one or more application-specific integrated circuits (ASICs), one or more state machines, digital circuitry, and/or any other suitable circuitry. In addition, each computing device may transmit and receive data over the communication network 22.

In some embodiments, each of the care management computing device 4 and the processing device(s) 10 may be a computer, a workstation, a laptop, a server such as a cloud-based server, or any other suitable device. In some embodiments, each of the processing devices 10 is a server that includes one or more processing units, such as one or more graphical processing units (GPUs), one or more central processing units (CPUs), and/or one or more processing cores. Each processing device 10 may, in some embodiments, execute one or more virtual machines. In some embodiments, processing resources (e.g., capabilities) of the one or more processing devices 10 are offered as a cloud-based service (e.g., cloud computing). For example, the cloud-based engine 8 may offer computing and storage resources of the one or more processing devices 10 to the care management computing device 4.

In some embodiments, each of the user computing devices 16, 18, 20 may be a cellular phone, a smart phone, a tablet, a personal assistant device, a voice assistant device, a digital assistant, a laptop, a computer, or any other suitable device. In some embodiments, the web server 6 hosts one or more network environments, such as an e-commerce network environment. In some embodiments, the care management computing device 4, the processing devices 10, and/or the web server 6 are operated by the network environment provider, and the user computing devices 16, 18, 20 are operated by users of the network environment. In some embodiments, the processing devices 10 are operated by a third party (e.g., a cloud-computing provider).

Although FIG. 1 illustrates three user computing devices 16, 18, 20, the network environment 2 may include any number of user computing devices 16, 18, 20. Similarly, the network environment 2 may include any number of the care management computing device 4, the web server 6, the processing devices 10, and/or the databases 14. It will further be appreciated that additional systems, servers, storage mechanism, etc. may be included within the network environment 2. In addition, although embodiments are illustrated herein having individual, discrete systems, it will be appreciated that, in some embodiments, one or more systems may be combined into a single logical and/or physical system. For example, in various embodiments, one or more of the care management computing device 4, the web server 6, the database 14, the user computing devices 16, 18, 20, and/or the router 24 may be combined into a single logical and/or physical system. Similarly, although embodiments are illustrated having a single instance of each device or system, it will be appreciated that additional instances of a device may be implemented within the network environment 2. In some embodiments, two or more systems may be operated on shared hardware in which each system operates as a separate, discrete system utilizing the shared hardware, for example, according to one or more virtualization schemes.

The communication network 22 may be a WiFi® network, a cellular network such as a 3GPP® network, a Bluetooth® network, a satellite network, a wireless local area network (LAN), a network utilizing radio-frequency (RF) communication protocols, a Near Field Communication (NFC) network, a wireless Metropolitan Area Network (MAN) connecting multiple wireless LANs, a wide area network (WAN), or any other suitable network. The communication network 22 may provide access to, for example, the Internet.

Each of the user computing devices 16, 18, 20 may communicate with the web server 6 over the communication network 22. For example, each of the user computing devices 16, 18, 20 may be operable to view, access, and interact with a website, such as an e-commerce website, hosted by the web server 6. The web server 6 may transmit user session data related to a user's activity (e.g., interactions) on the website. For example, a user may operate one of the user computing devices 16, 18, 20 to initiate a web browser that is directed to the website hosted by the web server 6. The user may, via the web browser, perform various operations such as searching one or more databases or catalogs associated with the displayed website, view data for elements associated with and displayed on the website, and click on interface elements presented via the website. The website may capture these activities as user session data, and transmit the user session data to the care management computing device 4 over the communication network 22. The website may also allow the user to interact with one or more of interface elements to perform specific operations, such as selecting one or more elements for further processing. In some embodiments, the web server 6 transmits user interaction data identifying interactions between the user and the website to the care management computing device 4.

In some embodiments, the care management computing device 4 may execute one or more models, processes, or algorithms, such as a machine learning model, deep learning model, statistical model, etc., to care management. The care management computing device 4 may transmit care management alerts or triggers to the web server 6 over the communication network 22, and the web server 6 may display interface elements associated with care management alerts on the website to the user (e.g., one or more associates of a care management panel assigned to the a respective member). For example, the web server 6 may display interface elements associated with care management alerts and/or member profiles to the user on a homepage, a catalog webpage, an item webpage, a window or interface of a chatbot, a search results webpage, or a post-transaction webpage of the website (e.g., as the user browses those respective webpages).

The care management computing device 4 is further operable to communicate with the database 14 over the communication network 22. For example, the care management computing device 4 may store data to, and read data from, the database 14. The database 14 may be a remote storage device, such as a cloud-based server, a disk (e.g., a hard disk), a memory device on another application server, a networked computer, or any other suitable remote storage. Although shown remote to the care management computing device 4, in some embodiments, the database 14 may be a local storage device, such as a hard drive, a non-volatile memory, or a USB stick. The care management computing device 4 may store interaction data received from the web server 6 in the database 14. The care management computing device 4 may also receive from the web server 6 user session data identifying events associated with browsing sessions, and may store the user session data in the database 14.

In some embodiments, the care management computing device 4 generates training data for a plurality of models (e.g., machine learning models, deep learning models, statistical models, algorithms, etc.) based on aggregation data, variant-level data, holiday and event data, recall data, historical user session data, search data, purchase data, catalog data, advertisement data for the users, etc. The care management computing device 4 and/or one or more of the processing devices 10 may train one or more models based on corresponding training data. The care management computing device 4 may store the models in a database, such as in the database 14 (e.g., a cloud storage database).

The models, when executed by the care management computing device 4, allow the care management computing device 4 to generate a member profile, assign a medical panel of one or more associates, and automatically generate various alerts based on identified risks and needs of respective member. For example, the care management computing device 4 may obtain one or more models from the database 14. The care management computing device 4 may then receive, in real-time from the web server 6, a care management event associated with a respective member in the population. In response to receiving care management event, the care management computing device 4 may execute one or more models to update the member profile, recompute risk scores associated with the member profile, update assign care management panel, and generate a care management alerts based on the member profile.

In some embodiments, the care management computing device 4 assigns the models (or parts thereof) for execution to one or more processing devices 10. For example, each model may be assigned to a virtual machine hosted by a processing device 10. The virtual machine may cause the models or parts thereof to execute on one or more processing units such as GPUs. In some embodiments, the virtual machines assign each model (or part thereof) among a plurality of processing units. Based on the output of the models, care management computing device 4 may generate risks scores associated with members in a population, stratify the member population into risk groups based on the risks scores, assign a medical panel to each member based on affiliation to one of the risk groups, and generate alerts based on the member profiles, including associated risk groups.

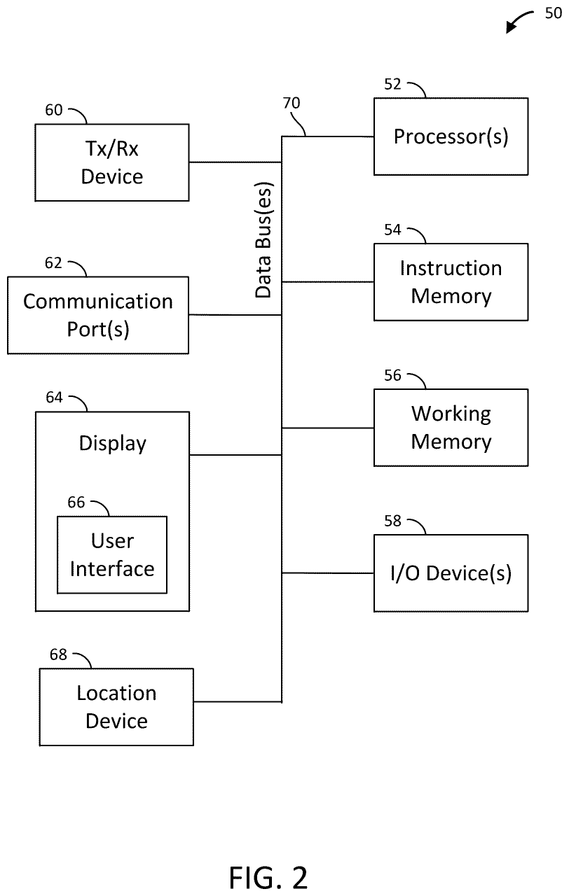

FIG. 2 illustrates a block diagram of a computing device 50, in accordance with some embodiments. In some embodiments, each of the care management computing device 4, the web server 6, the one or more processing devices 10, the workstation(s) 12, and/or the user computing devices 16, 18, 20 in FIG. 1 may include the features shown in FIG. 2. Although FIG. 2 is described with respect to certain components shown therein, it will be appreciated that the elements of the computing device 50 may be combined, omitted, and/or replicated. In addition, it will be appreciated that additional elements other than those illustrated in FIG. 2 may be added to the computing device.

As shown in FIG. 2, the computing device 50 may include one or more processors 52, an instruction memory 54, a working memory 56, one or more input/output devices 58, a transceiver 60, one or more communication ports 62, a display 64 with a user interface 66, and an optional location device 68, all operatively coupled to one or more data buses 70. The data buses 70 allow for communication among the various components. The data buses 70 may include wired, or wireless, communication channels.

The one or more processors 52 may include any processing circuitry operable to control operations of the computing device 50. In some embodiments, the one or more processors 52 include one or more distinct processors, each having one or more cores (e.g., processing circuits). Each of the distinct processors may have the same or different structure. The one or more processors 52 may include one or more central processing units (CPUs), one or more graphics processing units (GPUs), application specific integrated circuits (ASICs), digital signal processors (DSPs), a chip multiprocessor (CMP), a network processor, an input/output (I/O) processor, a media access control (MAC) processor, a radio baseband processor, a co-processor, a microprocessor such as a complex instruction set computer (CISC) microprocessor, a reduced instruction set computing (RISC) microprocessor, and/or a very long instruction word (VLIW) microprocessor, or other processing device. The one or more processors 52 may also be implemented by a controller, a microcontroller, an application specific integrated circuit (ASIC), a field programmable gate array (FPGA), a programmable logic device (PLD), etc.

In some embodiments, the one or more processors 52 are configured to implement an operating system (OS) and/or various applications. Examples of an OS include, for example, operating systems generally known under various trade names such as Apple macOS™, Microsoft Windows™, Android™, Linux™, and/or any other proprietary or open-source OS. Examples of applications include, for example, network applications, local applications, data input/output applications, user interaction applications, etc.

The instruction memory 54 may store instructions that are accessed (e.g., read) and executed by at least one of the one or more processors 52. For example, the instruction memory 54 may be a non-transitory, computer-readable storage medium such as a read-only memory (ROM), an electrically erasable programmable read-only memory (EEPROM), flash memory (e.g. NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. The one or more processors 52 may be configured to perform a certain function or operation by executing code, stored on the instruction memory 54, embodying the function or operation. For example, the one or more processors 52 may be configured to execute code stored in the instruction memory 54 to perform one or more of any function, method, or operation disclosed herein.

Additionally, the one or more processors 52 may store data to, and read data from, the working memory 56. For example, the one or more processors 52 may store a working set of instructions to the working memory 56, such as instructions loaded from the instruction memory 54. The one or more processors 52 may also use the working memory 56 to store dynamic data created during one or more operations. The working memory 56 may include, for example, random access memory (RAM) such as a static random access memory (SRAM) or dynamic random access memory (DRAM), Double-Data-Rate DRAM (DDR-RAM), synchronous DRAM (SDRAM), an EEPROM, flash memory (e.g. NOR and/or NAND flash memory), content addressable memory (CAM), polymer memory (e.g., ferroelectric polymer memory), phase-change memory (e.g., ovonic memory), ferroelectric memory, silicon-oxide-nitride-oxide-silicon (SONOS) memory, a removable disk, CD-ROM, any non-volatile memory, or any other suitable memory. Although embodiments are illustrated herein including separate instruction memory 54 and working memory 56, it will be appreciated that the computing device 50 may include a single memory unit configured to operate as both instruction memory and working memory. Further, although embodiments are discussed herein including non-volatile memory, it will be appreciated that computing device 50 may include volatile memory components in addition to at least one non-volatile memory component.

In some embodiments, the instruction memory 54 and/or the working memory 56 includes an instruction set, in the form of a file for executing various methods, such as methods for automated care management intervention, as described herein. The instruction set may be stored in any acceptable form of machine-readable instructions, including source code or various appropriate programming languages. Some examples of programming languages that may be used to store the instruction set include, but are not limited to: Java, JavaScript, C, C++, C#, Python, Objective-C, Visual Basic, .NET, HTML, CSS, SQL, NoSQL, Rust, Perl, etc. In some embodiments a compiler or interpreter is configured to convert the instruction set into machine executable code for execution by the one or more processors 52.

The input-output devices 58 may include any suitable device that allows for data input or output. For example, the input-output devices 58 may include one or more of a keyboard, a touchpad, a mouse, a stylus, a touchscreen, a physical button, a speaker, a microphone, a keypad, a click wheel, a motion sensor, a camera, and/or any other suitable input or output device.

The transceiver 60 and/or the communication port(s) 62 allow for communication with a network, such as the communication network 22 of FIG. 1. For example, if the communication network 22 of FIG. 1 is a cellular network, the transceiver 60 is configured to allow communications with the cellular network. In some embodiments, the transceiver 60 is selected based on the type of the communication network 22 the computing device 50 will be operating in. The one or more processors 52 are operable to receive data from, or send data to, a network, such as the communication network 22 of FIG. 1, via the transceiver 60.

The communication port(s) 62 may include any suitable hardware, software, and/or combination of hardware and software that is capable of coupling the computing device 50 to one or more networks and/or additional devices. The communication port(s) 62 may be arranged to operate with any suitable technique for controlling information signals using a desired set of communications protocols, services, or operating procedures. The communication port(s) 62 may include the appropriate physical connectors to connect with a corresponding communications medium, whether wired or wireless, for example, a serial port such as a universal asynchronous receiver/transmitter (UART) connection, a Universal Serial Bus (USB) connection, or any other suitable communication port or connection. In some embodiments, the communication port(s) 62 allows for the programming of executable instructions in the instruction memory 54. In some embodiments, the communication port(s) 62 allow for the transfer (e.g., uploading or downloading) of data, such as machine learning model training data.

In some embodiments, the communication port(s) 62 are configured to couple the computing device 50 to a network. The network may include local area networks (LAN) as well as wide area networks (WAN) including without limitation Internet, wired channels, wireless channels, communication devices including telephones, computers, wire, radio, optical and/or other electromagnetic channels, and combinations thereof, including other devices and/or components capable of/associated with communicating data. For example, the communication environments may include in-body communications, various devices, and various modes of communications such as wireless communications, wired communications, and combinations of the same.

In some embodiments, the transceiver 60 and/or the communication port(s) 62 are configured to utilize one or more communication protocols. Examples of wired protocols may include, but are not limited to, Universal Serial Bus (USB) communication, RS-232, RS-422, RS-423, RS-485 serial protocols, Fire Wire, Ethernet, Fibre Channel, MIDI, ATA, Serial ATA, PCI Express, T-1 (and variants), Industry Standard Architecture (ISA) parallel communication, Small Computer System Interface (SCSI) communication, or Peripheral Component Interconnect (PCI) communication, etc. Examples of wireless protocols may include, but are not limited to, the Institute of Electrical and Electronics Engineers (IEEE) 802.xx series of protocols, such as IEEE 802.11a/b/g/n/ac/ag/ax/be, IEEE 802.16, IEEE 802.20, GSM cellular radiotelephone system protocols with GPRS, CDMA cellular radiotelephone communication systems with 1×RTT, EDGE systems, EV-DO systems, EV-DV systems, HSDPA systems, Wi-Fi Legacy, Wi-Fi 1/2/3/4/5/6/6E, wireless personal area network (PAN) protocols, Bluetooth Specification versions 5.0, 6, 7, legacy Bluetooth protocols, passive or active radio-frequency identification (RFID) protocols, Ultra-Wide Band (UWB), Digital Office (DO), Digital Home, Trusted Platform Module (TPM), ZigBee, etc.

The display 64 may be any suitable display, and may display the user interface 66. The user interfaces 66 may enable user interaction with automated care management system. For example, the user interface 66 may be a user interface for an application of a network environment operator that allows a user to view and interact with the operator's website. In some embodiments, a user may interact with the user interface 66 by engaging the input-output devices 58. In some embodiments, the display 64 may be a touchscreen, where the user interface 66 is displayed on the touchscreen.

The display 64 may include a screen such as, for example, a Liquid Crystal Display (LCD) screen, a light-emitting diode (LED) screen, an organic LED (OLED) screen, a movable display, a projection, etc. In some embodiments, the display 64 may include a coder/decoder, also known as Codecs, to convert digital media data into analog signals. For example, the visual peripheral output device may include video Codecs, audio Codecs, or any other suitable type of Codec.

The optional location device 68 may be communicatively coupled to a location network and operable to receive position data from the location network. For example, in some embodiments, the location device 68 includes a GPS device configured to receive position data identifying a latitude and longitude from one or more satellites of a GPS constellation. As another example, in some embodiments, the location device 68 is a cellular device configured to receive location data from one or more localized cellular towers. Based on the position data, the computing device 50 may determine a local geographical area (e.g., town, city, state, etc.) of its position.

In some embodiments, the computing device 50 is configured to implement one or more modules or engines, each of which is constructed, programmed, configured, or otherwise adapted, to autonomously carry out a function or set of functions. A module/engine may include a component or arrangement of components implemented using hardware, such as by an application specific integrated circuit (ASIC) or field-programmable gate array (FPGA), for example, or as a combination of hardware and software, such as by a microprocessor system and a set of program instructions that adapt the module/engine to implement the particular functionality, which (while being executed) transform the microprocessor system into a special-purpose device. A module/engine may also be implemented as a combination of the two, with certain functions facilitated by hardware alone, and other functions facilitated by a combination of hardware and software. In certain implementations, at least a portion, and in some cases, all, of a module/engine may be executed on the processor(s) of one or more computing platforms that are made up of hardware (e.g., one or more processors, data storage devices such as memory or drive storage, input/output facilities such as network interface devices, video devices, keyboard, mouse or touchscreen devices, etc.) that execute an operating system, system programs, and application programs, while also implementing the engine using multitasking, multithreading, distributed (e.g., cluster, peer-peer, cloud, etc.) processing where appropriate, or other such techniques. Accordingly, each module/engine may be realized in a variety of physically realizable configurations, and should generally not be limited to any particular implementation exemplified herein, unless such limitations are expressly called out. In addition, a module/engine may itself be composed of more than one sub-modules or sub-engines, each of which may be regarded as a module/engine in its own right. Moreover, in the embodiments described herein, each of the various modules/engines corresponds to a defined autonomous functionality; however, it should be understood that in other contemplated embodiments, each functionality may be distributed to more than one module/engine. Likewise, in other contemplated embodiments, multiple defined functionalities may be implemented by a single module/engine that performs those multiple functions, possibly alongside other functions, or distributed differently among a set of modules/engines than specifically illustrated in the embodiments herein.

Care management is important for a number of reasons. First, it can help improve the quality of care for patients with chronic conditions. Studies have shown that care management can lead to reductions in hospital admissions, emergency room visits, and readmission rates. Second, care management can help improve the coordination of care for patients with chronic conditions. This can lead to better outcomes and lower costs. Third, care management can help educate and support patients and their caregivers. This can lead to improved self-management and better adherence to treatment plans. Fourth, care management can help to identify and address barriers to care. This can improve access to care and better health outcomes.

However, not all patients with chronic conditions are good candidates for care management. For example, patients may require different level, frequency, intensity of care, timing of engagement throughout the patient's tenure depending on clinical needs, access to care, patient's condition, care manager capacity, care management workflows, and/or any regulatory requirements. In addition to these factors, another important factor to consider is cost. Care management is costly and capacity is limited, and it is therefore desirable to consider the cost of care management when a system selects which patients to be enrolled in a care management program. Assigning care management resources efficiently improves reduces cost while also improves impact on the patient's health.

The described methods and systems identify and prioritize members of a managed population for care management services and support based on a number of factors using predictive models to optimize and improve care management interventions.

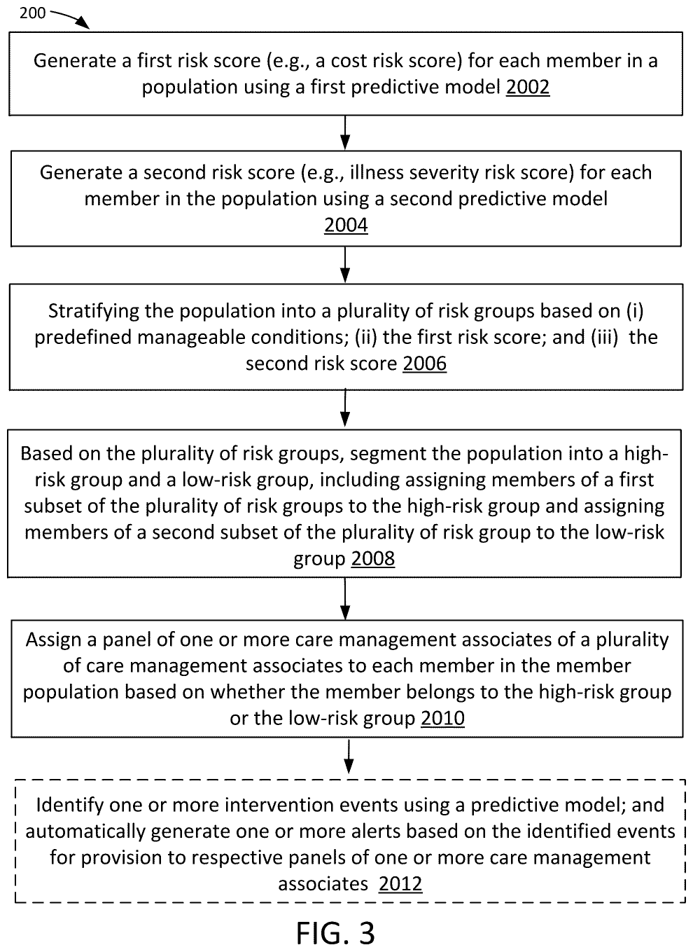

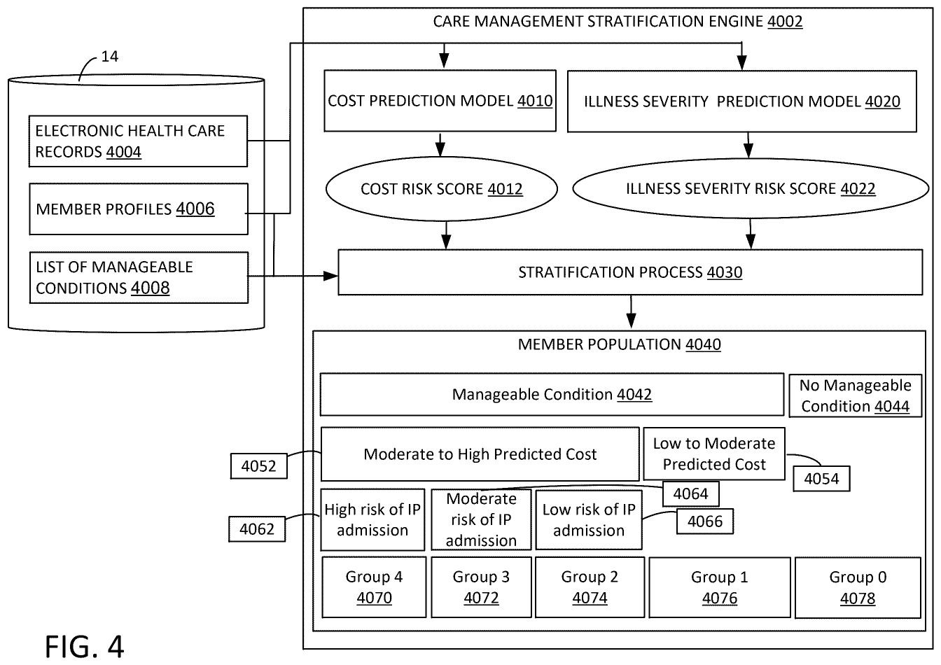

FIG. 3 is a flowchart illustrating a method 200 for generating automated care management intervention, in accordance with some embodiments. A first risk score (e.g., a cost risk score 4012 in FIG. 4) is generated (2002) for each member in a population using a first predictive model (e.g., cost prediction model 4010 in FIG. 4). In some embodiments, the population includes a group of people (e.g., patients or medically insured members) that have been preselected for analysis across multiple dimensions. In some embodiments, the first predictive model is a machine learning model that predicts the likelihood (e.g., probability) of medical costs for a respective member in the member population reaching a respective predetermined percentile (e.g., top 1%, 2%, 3%, 5%) or threshold amount (e.g., $10,000.00, $15,000.00, $20,000.00, $25,000.00, $30,000.00, $35,000.00, etc.) optionally within a respective time frame (e.g., within the next 1 week, 2 weeks, 1 month, 2 months, 3 months, 4 months, 6 months, 1 year, etc.). In some embodiments, the first predictive model is referred to as cost prediction model(s) (e.g., Table 1 for exemplary key variables of the cost prediction model). In some embodiments, data inputs or data sources to the first predictive model includes demographic information (e.g., age, gender, socioeconomic status, geographic location), clinical history (e.g., chronic conditions (e.g., diabetes, hypertension), previous hospitalizations, medication adherence, utilization patterns (e.g., emergency room visits in the past year, number of hospital readmissions, frequency of primary care visits), social determinants of health (e.g., housing stability, access to transportation, social support networks), health behaviors (e.g., tobacco use, physical activity levels, diet and nutrition habits), and others. In some embodiments, electronic health care records of the members of the population are used to supply the information for the data inputs to the first predictive model. In some embodiments, the first predictive model uses regression analysis to identify statistically significant relationships between historical data (e.g., demographics, clinical history, utilization patterns, social determinants of health, and health behaviors) and the associated healthcare costs. In some embodiments additional machine learning models are employed to improve the accuracy of predictions, such as such as decision trees or random forests. These algorithms can identify complex patterns in the data that may not be apparent through traditional regression analysis. In some embodiments, the first predictive model builds upon or is based on a number of machine learning models for regression, classification, and ranking. For example, the first predictive model builds upon or is based on a number of machine learning models that improve upon one another, including supervised machine learning, decision trees, ensemble learning, and/or gradient boosting. In some embodiments, a library that implements optimized distributed gradient boosting machine learning algorithms under the Gradient Boosting framework can be used to determine a cost risk score, such as XGBoost software library. In some embodiments, the model outputs a risk score (e.g., a cost risk score), or a probability estimate score for each patient or member in the population, indicating likelihood of incurring higher care management costs in the future. Table 1 below list a selected variables that are used in the cost prediction model, according to one embodiment.

TABLE 1

| | |
|---|---|
| | Feature |
| Selected Features | Description |

NUM_MBR_LNGTD
NUM_MBR_AGE
NUM_ALLCLMS_TTLMDCLCLMS_2Y_PDAMT
NUM_CCS_256_DSL
NUM_C3N_0945_DSL
NUM_MBR_LATD
NUM_C1N_046_DSL
NUM_UM_SRVCMED_2Y_DSL
NUM_ELXREADMITSCORE
NUM_CLNCL_ASCALL_1Y_AMT
NUM_ALLCLMS_TTLMDCLCLMS_3M_ALWAMT
NUM_HLTH_HCUP_DSL
NUM_C3N_0946_DSL
NUM_ALLCLMS_TTLMDCLCLMS_1Y_PDAMT
NUM_C3N_0407_DSL

In one example, the first predictive model may predict the likelihood that a respective member in a qualifying member population would have medical costs more than a threshold amount in the near future, such as $36,309.24. For example, a member in the qualifying population is defined to have the model target outcome if the total sum of paid amount through claims which have service starting date within the next 6 months and were received within the next 9 months (e.g., 3-month buffer) is greater than or equal to $36,309.24. For example, the respective cut-off value of $36,309.24 is selected because it corresponds to the top 2nd percentile of the sum of paid amount. The first predictive model (e.g., the cost prediction model) determines the number of members in the member population that have the model target outcome (e.g., 10,388 members out of 515,261 qualifying members).

A second risk score (e.g., an illness severity risk score) is generated (2004) for each member in the population using a second predictive model different from the first predictive model. In some embodiments, the second score represents a likelihood (e.g., a probability) of an inpatient admission (e.g., admitting a respective patient of the member population to a medical facility or a hospital). For example, the second predictive model predicts the likelihood of a medical surgery or inpatient admission (e.g., for behavioral or mental health condition or other reasons) within a respective time frame (e.g., within the next 1 week, 2 weeks, 1 month, 2 months, 3 months, 4 months, 6 months, 1 year, etc.). In some embodiments, the second predictive model is a logistic regression model. For example, the second predictive model uses logistic regression to analyze relationships between the input variables and the binary outcome of inpatient admission (1 for admission, 0 for no admission). The coefficients derived from the regression analysis indicate the strength and direction of these relationships. The model assigns a risk score to each patient based on the logistic regression results. The risk score represents the estimated probability of inpatient admission. A threshold is set to categorize patients into different risk levels. For example, (i) low risk corresponds to a probability of admission of less 0.2; (ii) moderate risk corresponds to a probability of admission between 0.2 and 0.8; and (iii) high risk corresponds to probability of more than or equal to 0.8. The model outputs a risk score for each patient, indicating their likelihood of being admitted to the hospital within a specified timeframe. In some embodiments, the second predictive model predicts the severity of illness for a patient. Predicting the severity of illness can be a crucial aspect of care management, especially for individuals with complex or chronic conditions. The second predictive model in this context aim to assess the likely progression and severity of a patient's health status, enabling care managers to tailor interventions accordingly. Example data inputs for the predictive model that predicts illness severity includes, but is not limited to, clinical indicators (e.g., current symptoms and their severity, vital signs (e.g., heart rate, respiratory rate); laboratory results (e.g., blood tests, and/or imaging), disease-specific biomarkers; chronic conditions (e.g., pre-existing chronic diseases (e.g., diabetes, heart failure, and/or chronic kidney disease), and/or disease-specific markers and indicators); medication history (e.g., current medications and dosages and/or medication adherence history); functional status (e.g., activities of daily living (ADL) assessment and/or mobility and functional independence); social determinants of health (e.g., living conditions and stability, access to a support system, and/or other socioeconomic factors). In some embodiments, the second predictive model can use machine learning models, such as gradient boosting or neural networks, that analyze complex relationships within the input data to predict the severity of illness. These machine learning models capture non-linear patterns and interactions among multiple variables. In some embodiments, these models perform feature important analysis. For example, these machine learning models identify the most influential features contributing to the prediction of severity. Such an analysis can help care managers understand which factors play a significant role in assessing the patient's health status. In some embodiments, the patient population is stratified based on the identified risk. For example, patients or members are categorized into different risk levels based on the predicted severity of illness. For example, (i) low severity category corresponds to minimal expected progression of illness; (ii) moderate severity corresponds to moderate likelihood of worsening health; and (iii) high severity corresponds to high risk of significant deterioration.

In some embodiments, illness severity predictions can be used for a variety of health care management services. For example, illness severity predictions provided by the prediction models can be used to generate tailored care plans. For example, care managers use the severity predictions to tailor care plans based on the specific needs and risks of the patient. High-severity patients may require more intensive interventions and frequent monitoring. In another example, illness severity predictions provided by the prediction models can be used to more efficiently allocate resources. For example, the care management system or care mangers can allocate resources more efficiently by directing more intensive interventions and support to patients at higher risk of severe illness. In another example, illness severity predictions provided by the prediction models can be used for patient education and engagement. For example, predictions of severity can be used to educate patients about their health conditions and motivate them to actively participate in their care plans, especially for those at higher risk. In some embodiments, the prediction models are continuously updated as new data becomes available. This allows for adjustments to care plans based on the evolving health status of the patient. In some embodiments, illness severity predictions provided by the prediction models can be used to foster and enable interdisciplinary collaboration. For example, care managers that have access to the outputs of the predictive models can collaborate with other healthcare professionals to ensure a holistic approach to managing the patient's health. This may involve coordination with physicians, nurses, therapists, and social workers. In some embodiments, example data inputs for the second predictive model include, but are not limited to, demographic information (e.g., age, gender, race/ethnicity, socioeconomic status), clinical history (e.g., chronic conditions (e.g., diabetes, heart failure, COPD), medication adherence, previous hospitalizations), utilization patterns (e.g., emergency room visits in the past year, number of primary care visits, specialist visits), health status (e.g., vital signs (e.g., blood pressure, heart rate), recent laboratory results (e.g., blood glucose levels)), social determinants of health (e.g., living conditions, access to transportation, social support networks).

In some embodiments, the first predictive and the second predictive model use multiple data sources including, but not limited to, demographics, clinical historical data, care management history (e.g., prior interactions with care management, prescribed and/or performed treatments, and/or utilized or unutilized care management services) performed lab tests and respective lab results, historical data regarding pharmacy use (e.g., fulfilled and/or unfulfilled prescriptions, history of chronic and ambulatory sensitive conditions, overall cost and utilization of care services and benefits, authorization requests, and others.

The population is stratified (2006) into a first plurality of risk groups based on: (i) predefined manageable medical conditions (e.g., diagnosis groups that clinicians labeled or identified as manageable by one or more care management programs, treatments, or interventions); (ii) the first risk score (e.g., cost risk score); and (iii) the second risk score (e.g., illness severity risk score). For example, the member population may be divided into a number of risk groups (e.g., two, three, four, five, or more risk groups). In some embodiments, the population is first stratified based on a predefined list of manageable medical conditions, then the population is further stratified based on the cost risk score, and finally the stratified population is further stratified based on the illness severity risk score. For example, the three-level stratification is illustrated in FIG. 4 (e.g., stratified population 4040).

In some embodiments, the list of manageable conditions based on which a first level stratification is performed is provided by a team of clinicians. Care management often focuses on individuals with complex or chronic health conditions that require ongoing support and coordination of care. The specific conditions determined to be “manageable” may vary depending on the healthcare setting, available resources, and the expertise of the care management team. An example list of manageable conditions commonly addressed in care management includes, but is not limited to, diabetes mellitus (e.g., Type 1 diabetes and Type 2 diabetes); cardiovascular conditions (e.g., hypertension (high blood pressure), coronary artery disease, and/or heart failure); chronic respiratory diseases (e.g., chronic obstructive pulmonary disease (COPD), or asthma); kidney diseases (e.g., chronic kidney disease (CKD)); neurological conditions (e.g., stroke, Parkinson's disease, or multiple sclerosis); mental health conditions (e.g., depression, anxiety disorders, bipolar disorder, or schizophrenia); autoimmune diseases (e.g., rheumatoid arthritis or systemic lupus erythematosus (SLE)); gastrointestinal conditions (e.g., inflammatory bowel disease (IBD) or gastroesophageal reflux disease (GERD)); endocrine disorders (e.g., thyroid disorders (e.g., hypothyroidism, hyperthyroidism)); cancer survivorship (e.g., post-treatment support and coordination); palliative care (e.g., individuals with serious or life-limiting illnesses); geriatric conditions (e.g., frailty or polypharmacy management in the elderly); pain management (e.g., chronic pain conditions); HIV/AIDS (e.g., management of individuals living with HIV/AIDS); obesity and weight Management (e.g., support for weight loss and lifestyle changes); maternal and child Health (e.g., high-risk pregnancy or pediatric chronic conditions); post-operative care (e.g., coordination of care after surgeries); complex medication regimens (e.g., individuals taking multiple medications; transitional care (e.g., support during transitions between care settings (e.g., hospital to home)); or complex wound care (e.g., individuals with chronic wounds or conditions requiring specialized care).

Based on the plurality of risk groups, the population is segmented (2008) (e.g., or further stratified) into a high-risk group and a low-risk group. For example, members of a first subset of the plurality of risk groups (e.g., group 0, 1, 2) are assigned to the high-risk group and members of a second subset of the plurality of risk groups are assigned to the low-risk group (e.g., groups 3, 4, 5).

In some embodiments, members of a respective risk group of the first plurality of risk groups are assigned priority based on an engagement score determined by a third predictive model. Predicting the level of patient engagement is crucial for effective care management, as engaged patients are more likely to actively participate in their care plans and achieve better health outcomes. In some embodiments, members within a group of the first plurality of groups are prioritized for care management services and outreach based on an engagement score determined for each member. For example, members with a higher engagement score are assigned higher priority than members with a lower engagement score.

In some embodiments, the third predictive model (e.g., also referred to as engagement prediction model) assess the level of patient engagement of each member in the managed population, and outputs an engagement score for each managed member in the population. In some embodiments, the third predictive model may use logistic regression or classification algorithms (e.g., decision trees, random forests) to analyze the relationships between the input variables and the binary outcome of patient engagement (e.g., high or low engagement). In some embodiments, the engagement prediction model can identify the top or key factors that contribute most significantly to patient engagement. In some embodiments, the member population can be categorized into different segments based on predicted level of engagement of each patient, such as: Highly Engaged, Moderately Engaged, or Low Engagement. In some embodiments, the third predictive model outputs a predictive score or category indicating the estimated level of patient engagement of each managed member for future care management activities. In some embodiments, example data inputs of the third predictive model include, but are not limited to, demographic Information (e.g., age, gender, education level, or employment status); health literacy and numeracy (e.g., patient's ability to understand and interpret health information); previous engagement patterns (e.g., history of attending medical appointments, adherence to previous care plans, or participation in health education programs); communication preferences (e.g., preferred communication channels (e.g., phone, email, in-person), language preferences; social determinants of health (e.g., family support, financial stability, or access to transportation); health status (e.g., chronic conditions and comorbidities, recent hospitalizations, or emergency room visits); patient activation measure (PAM) (e.g., a standardized tool assessing a patient's knowledge, skill, and confidence for managing their health).

A panel of one or more care management associates of a plurality of care management associates is assigned (2010) to each member in the member population based on whether the member belongs to the high-risk group or the low-risk group. In some embodiments, the plurality of care management associates are segmented into a high-risk group and a low-risk group. In some embodiments, assessing the quality or level of service provided by a care management associate is critical for optimizing healthcare outcomes and ensuring a positive patient experience. In some embodiments, predictive models are used to identify factors associated with high-quality care management. In some embodiments, a fourth predictive model is used assess the quality of service provided by a care management associate, and outputs a respective score corresponding or representing the estimated quality of services (e.g., or level of risk associated with the services provide by the respective associate). In some embodiments, the fourth predictive model outputs (or provides) a predictive score or category indicating the estimated level of service quality for each care management associate. In some embodiments, the service quality score that is outputted by the fourth predictive model (e.g., also referred to as service quality prediction model) is used to segment a plurality of care management associates (e.g., medical staff, nurses, social workers, or other providers of care management services) into categories, such as high-risk group or the low-risk group. For example, care management associates can be categorized into different segments based on their predicted level of service quality (e.g., High-Quality Service, Moderate-Quality Service, or Low-Quality Service). In some embodiments, the service quality prediction model (e.g., or collection of models) may use regression analysis or classification machine learning models (e.g., logistic regression, random forests) to analyze the relationships between the input variables and the outcome of service quality (e.g., high, moderate, low). In some embodiments, the service quality prediction model(s) identify factors that contribute most significantly to service quality. Accordingly, the service quality prediction model(s) identify key performance indicators. In some embodiments, example data inputs of the fourth predictive model include, but are not limited to, care management workflow data (e.g., number of patient interactions, frequency and type of communication (e.g., phone calls, emails), or documentation completeness); patient satisfaction surveys (e.g., responses to surveys regarding the patient's perception of the care management associate's service); adherence to care plans (e.g., evaluation of whether patients follow prescribed care plans, medication adherence rates); timeliness of responses (e.g., average response time to patient inquiries or issues, timeliness of care plan adjustments based on changing patient needs); interdisciplinary collaboration (e.g., collaboration with other healthcare providers and professionals, or attendance and participation in care team meetings); utilization of resources (e.g., appropriateness of resource allocation for patient needs); and/or efficiency in coordinating ancillary services (e.g., social services, rehabilitation).

In some embodiments, automated alerts or triggers are utilized to initiate care management services. These alerts are generated based on predefined criteria and/or machine learning models that identify situations requiring attention from a care management associate. In some embodiments, one or more intervention events are identified (2012) by a predictive model. One or more alerts (e.g., triggers) are automatically generated based on the identified one or more interventions events. In some embodiments, predictive model(s) can analyze patient data, including clinical and utilization history, to identify individuals at high risk for adverse events or hospitalizations. When a patient meets specific criteria indicating increased risk, an alert can be triggered to initiate care management interventions. Monitoring medication adherence is crucial for patients with chronic conditions. In some embodiments, automated systems can generate alerts when a patient misses doses or demonstrates inconsistent adherence patterns, prompting care managers to intervene and address potential issues. In some embodiments, automated alerts can be set up to notify care management teams when patients receive abnormal laboratory or diagnostic test results. This facilitates timely follow-up and adjustment of care plans as needed. In some embodiments, automated systems can identify care gaps, such as missed vaccinations or preventive screenings. Alerts are then generated to prompt care managers to engage with patients and facilitate the necessary interventions to close these gaps in care. In some embodiments, following hospital discharges, automated alerts can notify care management teams to initiate post-discharge follow-up and coordination to prevent readmissions and ensure a smooth transition to the community setting. In some embodiments, patient-reported data, collected through surveys or digital health tools, can trigger alerts when patients report worsening symptoms or a decline in their health status. Care managers can use this information to initiate timely interventions. In some embodiments, automated systems can generate alerts when patients miss scheduled appointments. Care managers can follow up to understand the reasons for non-attendance and address any barriers to engagement. In some embodiments, alerts can be triggered when patients visit the emergency room frequently, signaling the need for more intensive care management to address underlying issues and prevent unnecessary emergency department visits. In some embodiments, predictive models can identify patients at high risk of hospital readmission. Automated alerts can then notify care management teams to implement targeted interventions to reduce the likelihood of readmission. In some embodiments, predictive models may identify patients with high risk of exceeding maximum out of pocket limit. Automated alerts can then notify care management teams to engage with the patients as financial barrier is significantly reduced for such patients. In some embodiments, patient-initiated requests for assistance or support, whether through a helpline or a digital platform, can trigger alerts for care management involvement. In some embodiments, these automated alerts serve as proactive tools to help care management teams prioritize their efforts, address potential issues early, and provide timely and targeted support to patients.

In some embodiments, various predictive models can be employed to generate alerts for care management teams. In some embodiments, the choice of model that is employed depends on the specific use case and the type of data available. In some embodiments, logistic regression can be used for binary classification problems. For example, logistic regression could be employed to predict the likelihood of a patient being readmitted to a hospital within a certain timeframe. In some embodiments, random forests may be used for both classification and regression tasks. For example, random forests can be used to identify patterns in patient data that may lead to specific outcomes or events. In some embodiments, support vector machines (SVM) can be used for classification tasks. SVMs can be effective when dealing with high-dimensional data. In some embodiments, deep learning models, such as artificial neural networks, can be employed for complex tasks like predicting disease progression or identifying anomalies in patient data. In some embodiments, time series models, such as ARIMA (Auto Regressive Integrated Moving Average), can be applied to predict future trends in patient health based on historical data. In some embodiments, anomaly detection models can be used. For example, isolation forests can be used for detecting anomalies or outliers in datasets. This can be valuable for identifying unusual patterns in patient data that may require immediate attention. Another model for anomaly detection, One-Class SVM is trained on normal data and can identify instances that deviate significantly from the norm. In some embodiments, survival analysis models can be used. For example, Cox Proportional-Hazards Model can be used for predicting the time until a particular event (e.g., readmission or progression of a disease). In some embodiments, Natural Language Processing (NLP) Models, such as text mining and sentiment analysis can be used for analyzing unstructured data such as clinical notes and patient communications. NLP can be applied to detect early signs of patient deterioration or changes in mental health. In some embodiments, the predictive models can be combined with rule-based systems. For example, expert systems can be used where domain knowledge is incorporated into rule-based systems. Rule-based Systems can help generate alerts based on specific conditions or combinations of factors. In some embodiments, the model(s) identify situations requiring attention from a care management associate and also has an increased likelihood of driving action and potential engagement from a member. For example, based on engagement from previous alerts or triggers (e.g., historical data) and profile of members, the model(s) continuously learns and optimizes which triggers to act on given historical outcomes by members of similar profiles.

FIG. 4 is a block diagram illustrating various portions of a care management stratification engine 4002, in accordance with some embodiments. Cara management stratification engine 4002 uses a list of manageable conditions 4008, cost risk score 412, and illness severity risk score 4022 to stratify the member population 4040 into multiple risk groups (e.g., group “0” 4078, group “1” 4076, group “2” 4074, group “3” 4072, and group “4” 4070).

In FIG. 4, one embodiment of a data source is illustrated corresponding to database 14. In some embodiments, database 14 includes electronic health care records 4004 that are available for the managed members that have member profiles (e.g., that have a profile included in member profiles 4006). In some embodiments, electronic health care records 4004 are digital versions of patients' paper charts. For example, a record of the electronic health care records 4004 contain a patient's medical history, diagnoses, medications, treatment plans, immunization dates, allergies, radiology images, and/or laboratory test results. electronic health care records 4004 facilitate the sharing of information among healthcare providers, supporting coordinated and comprehensive care. In some embodiments, database 14 includes member profiles 4006. An example member profile is illustrated below in Table 2.

TABLE 2

| Name: | Jane Doe | |
|---|---|---|
| Date of Birth: | Jan. 15, 1965 | |
| Gender: | Female | |
| Contact | Address: 123 Main Street, Anytown, USA | |
| Information: | Phone: (555) 555-1234 | |
| | Email: jane.doe@email.com | |
| Medical | Primary Diagnosis: | Type 2 Diabetes |
| History: | | Mellitus |
| | Other Chronic Conditions: | Hypertension |
| | | Hyperlipidemia |
| | Previous Surgeries: | None reported |
| Medications | Metformin (1000 mg, twice daily) | |
| | Lisinopril (10 mg, once daily) | |
| | Atorvastatin (20 mg, once daily) | |
| Allergies | No known allergies | |
| Immunizations | Up-to-date on routine vaccinations | |

| | |
|---|---|
| Recent Lab | Hemoglobin A1c: 7.2% (within the last 3 months) |
| Results | Blood Pressure: 130/80 mmHg |
| | LDL Cholesterol: 90 mg/dL |
| Recent | None reported in the last year |
| Hospitalizations | |
| Social | Living alone |
| Determinants | Limited financial resources |
| of Health: | Limited transportation options |
| Behavioral | Reports occasional feelings of anxiety and stress |
| Health: | No history of diagnosed mental health disorders |
| Functional | Independent in activities of daily living |
| Status | Exercises regularly (30 minutes of walking, 5 |
| | times a week) |
| Health | Adheres to prescribed medications |
| Behaviors: | Follows a diabetic diet plan |
| | Monitors blood glucose levels regularly |
| Care Team: | Primary Care Physician: Dr. John Smith |
| | Endocrinologist: Dr. Susan Jones |
| | Pharmacist: Mark Johnson, PharmD |
| | Registered Dietitian: Sarah Thompson, RD |

In some embodiments, database 14 includes a list of manageable conditions 4008. In some embodiments, the conditions on the list of manageable conditions may be selected by clinicians. In some embodiments, the electronic health care records 4004 and the member profiles 4006 are provided as data input to the cost prediction model 4010 and the illness severity prediction model 4020. In some embodiments, the electronic health care records 4004 and the member profiles 4006 include information or are supplemented with additional data sources that include, but are not limited to, demographics, clinical historical data, care management history (e.g., prior interactions with care management, prescribed and/or performed treatments, and/or utilized or unutilized care management services) performed lab tests and respective lab results, historical data regarding pharmacy use (e.g., fulfilled and/or unfulfilled prescriptions, history of chronic and ambulatory sensitive conditions, overall cost and utilization of care services and benefits, authorization requests, and others. In some embodiments, the data sources that are provided to the to the cost prediction model 4010 and the illness severity prediction model 4020 may be stored in different databases or the same database 14.

In some embodiments, the stratification process 4030 receives as input member population 4040 and the list of manageable conditions 4008. Member population 4040 includes identifying information of managed members, such as members that have member profiles 4006 in database 14. In some embodiments, members included in the member population are a subset of or include all of the members with member profiles 4006. In some embodiments, the stratification process 4030 categorizes the members in the member population 4040 into different segments based on whether each members has a manageable condition or does not have a manageable conditions. For example, members in the member population 4040 are categorized in segment 4042 that includes members with one or more manageable conditions and segment 4044 that includes members without any manageable conditions.

In some embodiments, cost prediction model 4010 generates cost risk score 4012 for each member in the member population 4040 based on the electronic health care records 4004 and the member profiles 4006 (and other data sources not illustrated in database 14). In some embodiments, the stratification process 4030 categorizes the members in the member population 4040 into different segments based on cost risk score 4012. For example, the members in segment 4042 are categorized in (i) segment 4052 that includes members with moderate to high predicted cost and (ii) segment 4054 that includes members with low to moderate predicted cost, based on the cost risk score 4012. In some embodiments, thresholds are defined to specify cost risk score 4012 that is low, moderate, or high. In some embodiments, the thresholds maybe predetermined or maybe adjusted by a machine learning model.

In some embodiments, illness severity risk prediction model 4020 generates illness severity risk score 4022 for each member in the member population 4040 based on the electronic health care records 4004 and the member profiles 4006 (and other data sources not illustrated in database 14). In some embodiments, the stratification process 4030 categorizes the members in the member population 4040 into different segments based on illness severity risk score 4022. For example, the members in segment 4052 are categorized in (i) segment 4062 that includes members with high risk of inpatient admission (e.g., hospitalization); (ii) segment 4064 that includes members with moderate risk of inpatient admission; and (iii) segment 4066 that includes members with high low of inpatient admission. In some embodiments, thresholds are defined to specify illness severity risk score 4022 that is low, moderate, or high. In some embodiments, the thresholds maybe predetermined or maybe adjusted by a machine learning model.

In some embodiments, multiple risk groups are formed (e.g., optionally by the stratification process 4030) based on the segmented member population 4040 that includes segments 4042, 4044, 4052, 4054, 4062, 4064, and 4066. For example, members in segment 4062 are assigned to group “4” 4070 that include members with one or more manageable conditions (e.g., members included in segment 4042) that also have moderate to high risk of predicted cost (e.g., members included in segment 4052) and also have high risk of inpatient admission (e.g., members included in segment 4062). Members in segment 4064 are assigned to group “3” 4072 that include members with one or more manageable condition (e.g., members included in segment 4042) that also have moderate to high risk of predicted cost (e.g., members included in segment 4052) and have moderate risk of inpatient admission (e.g., members included in segment 4062). Members in segment 4066 are assigned to group “2” 4074 that include members with one or more manageable condition (e.g., members included in segment 4042) that also have moderate to high risk of predicted cost (e.g., members included in segment 4052) and have low risk of inpatient admission (e.g., members included in segment 4062). Members in segment 4054 are assigned to group “1” 4076 that include members with one or more manageable condition (e.g., members included in segment 4042) and have low to moderate predicted cost (e.g., members included in segment 4054). Finally, members in segment 4044 are assigned to group “0” 4078 that include members with no manageable condition (e.g., members included in segment 4044). In some embodiments, the stratification process 4030 may segment the member population 4040 in different segments. In some embodiments, different risk groups may be formed based on the segments 4042, 4044, 4052, 4054, 4062, 4064, and 4066.

In some embodiments, the stratification process 4030 stratifies members across dimensions including engagement level, cost share, utilization events, regulatory requirements, changes in health status, gaps in care, thereby allow case management associates to deploy resources appropriately within high risk and low risk member panels to develop specific member tailored interventions. In some embodiments, the stratification process 4030 using a variety of predictive models improves and/or optimizes care management resource allocation and health outcomes for patients.

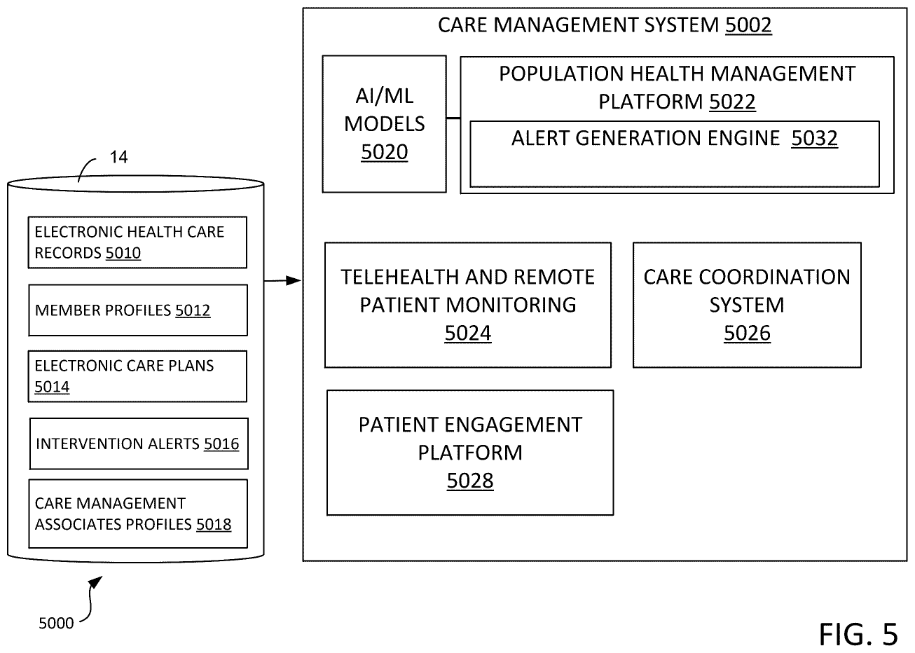

FIG. 5 is a block diagram 5000 illustrating various portions of a care management system 5002, in accordance with some embodiments. In some embodiments, care management system 5002 includes various systems that automate and improve care management services. For example, care management system 5002 includes various information systems, data analytics, and communication systems to streamline and improve various aspects of care management. For example, care management system 5002 has access to one or more database(s) 14 that includes various data sources in electronic form. For example, electronic health care records 5010 and member profiles 5012 are available for each managed member in a member population. In some embodiments, electronic care plans 5014 and intervention alerts 5016 are available for members in the member population. In some embodiments, database 14 includes profiles 5018 for care management associates.

In some embodiments, care management system 5002 includes a number of artificial intelligence/machine learning (AI/ML) models 5020. For example, AI/ML models 5020 include risk stratification models (e.g., cost risk prediction model 4010 and illness severity prediction model in FIG. 4). In one embodiment, AI/ML models 5020 include other stratification models, e.g., models that stratify the medical associates in risk groups (e.g., as described with reference to FIG. 3). In some embodiments, AI/ML models 5020 includes various predictive models can be employed to generate alerts for care management teams (e.g., as described with reference to FIG. 3).

In some embodiments, care management system 5002 includes a population health management platform 5022 that uses data analytics to identify and manage the health needs of specific patient populations. In some embodiments, population health management platform 5022 may use AI/ML models 5020 to continuously evaluate the needs and costs of managed members. In some embodiments, the population health management platform 5022 may include care management stratification engine 4003 in FIG. 4. The population health management platform 5022 helps care managers proactively address the needs of individuals with chronic conditions, track patient outcomes, and identify opportunities for intervention. In some embodiments, The population health management platform 5022 may include an alert generation engine 5032 powered by AI/ML models 5020. In some embodiments, alert generation engine 5032 automatically generate alerts or triggers using predictive models (e.g., as described with reference to FIG. 3).

In some embodiments, care management system 5002 includes telehealth and remote patient monitoring system 5024. In some embodiments, the telehealth and remote patient monitoring system 5024 allow for remote consultations between healthcare providers and patients. the telehealth and remote patient monitoring system 5024 monitors patients' health in real-time, allowing for early detection of issues and timely intervention. The telehealth and remote patient monitoring system 5024 is particularly beneficial for individuals with chronic conditions. In some embodiments, care management system 5002 includes care coordination system 5026 that helps healthcare teams coordinate and manage patient care more effectively. The care coordination system 5026 include features for scheduling appointments, tracking care plans, and facilitating communication among members of the care team. In some embodiments, care management system 5002 includes patient engagement platform 5028 that engages patients in their care by providing educational resources, appointment reminders, and tools for self-monitoring. patient engagement platform 5028 can empower individuals to take an active role in managing their health.

### Improvement Language

It will be appreciated that cost risk predictions, illness severity risks predictions, and intervention events predictions as disclosed herein, particularly on large datasets intended to be used population stratification and automated alert generation for care interventions, is possible with the aid of computer-assisted machine-learning algorithms and techniques, such as cost risk prediction model, illness severity risk prediction model, and/or intervention events prediction models. In some embodiments, machine learning processes including prediction model, illness severity risk prediction model, and/or intervention events prediction models are used to perform operations that cannot practically be performed by a human, either mentally or with assistance, such as predicting medical cost, illness severity, and events that require health care interventions of each patient in a large populations of managed members. It will be appreciated that a variety of machine learning techniques can be used alone or in combination to generate cost risk scores, illness severity risk scores, and/or automated alerts.

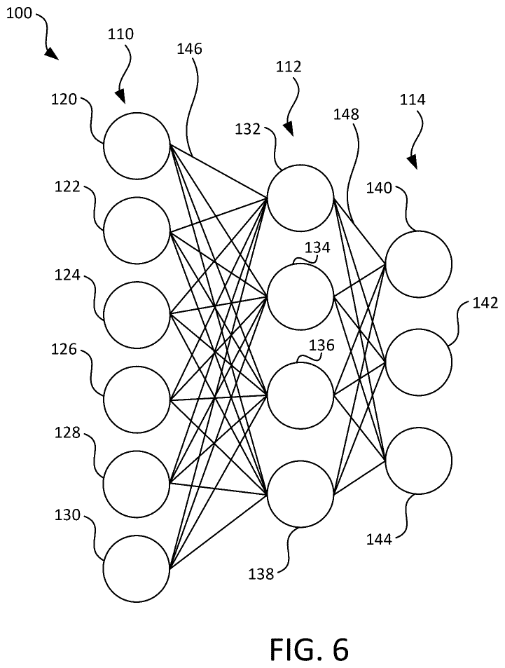

FIG. 6 illustrates an artificial neural network 100, in accordance with some embodiments. Alternative terms for “artificial neural network” are “neural network,” “artificial neural net,” “neural net,” or “trained function.” The neural network 100 comprises nodes 120-144 and edges 146-148, wherein each edge 146-148 is a directed connection from a first node 120-138 to a second node 132-144. In general, the first node 120-138 and the second node 132-144 are different nodes, although it is also possible that the first node 120-138 and the second node 132-144 are identical. For example, in FIG. 3 the edge 146 is a directed connection from the node 120 to the node 132, and the edge 148 is a directed connection from the node 132 to the node 140. An edge 146-148 from a first node 120-138 to a second node 132-144 is also denoted as “ingoing edge” for the second node 132-144 and as “outgoing edge” for the first node 120-138.

The nodes 120-144 of the neural network 100 may be arranged in layers 110-114, wherein the layers may comprise an intrinsic order introduced by the edges 146-148 between the nodes 120-144 such that edges 146-148 exist only between neighboring layers of nodes. In the illustrated embodiment, there is an input layer 110 comprising only nodes 120-130 without an incoming edge, an output layer 114 comprising only nodes 140-144 without outgoing edges, and a hidden layer 112 in-between the input layer 110 and the output layer 114. In general, the number of hidden layer 112 may be chosen arbitrarily and/or through training. The number of nodes 120-130 within the input layer 110 usually relates to the number of input values of the neural network, and the number of nodes 140-144 within the output layer 114 usually relates to the number of output values of the neural network.

In particular, a (real) number may be assigned as a value to every node 120-144 of the neural network 100. Here,

$${x}_{i}^{( n )}$$

denotes the value of the i-th node 120-144 of the n-th layer 110-114. The values of the nodes 120-130 of the input layer 110 are equivalent to the input values of the neural network 100, the values of the nodes 140-144 of the output layer 114 are equivalent to the output value of the neural network 100. Furthermore, each edge 146-148 may comprise a weight being a real number, in particular, the weight is a real number within the interval [−1, 1], within the interval [0, 1], and/or within any other suitable interval. Here,

$${w}_{i , j}^{( m , n )}$$

denotes the weight of the edge between the i-th node 120-138 of the m-th layer 110, 112 and the j-th node 132-144 of is defined for the weight the n-th layer 112, 114. Furthermore, the abbreviation

$${w}_{i , j}^{( n )}$$

is defined for the weight

$${w}_{i , j}^{( n , n + 1 )} .$$

In particular, to calculate the output values of the neural network 100, the input values are propagated through the neural network. In particular, the values of the nodes 132-144 of the (n+1)-th layer 112, 114 may be calculated based on the values of the nodes 120-138 of the n-th layer 110, 112 by

$${x}_{j}^{( n + 1 )} = f ( {\sum }_{i} {x}_{i}^{( n )} \cdot {w}_{i , j}^{( n )} )$$

Herein, the function f is a transfer function (another term is “activation function”). Known transfer functions are step functions, sigmoid function (e.g., the logistic function, the generalized logistic function, the hyperbolic tangent, the Arctangent function, the error function, the smooth step function) or rectifier functions. The transfer function is mainly used for normalization purposes.

In particular, the values are propagated layer-wise through the neural network, wherein values of the input layer 110 are given by the input of the neural network 100, wherein values of the hidden layer(s) 112 may be calculated based on the values of the input layer 110 of the neural network and/or based on the values of a prior hidden layer, etc.

In order to set the values

$${w}_{i , j}^{( m , n )}$$

for the edges, the neural network 100 has to be trained using training data. In particular, training data comprises training input data and training output data. For a training step, the neural network 100 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 100 (backpropagation algorithm). In particular, the weights are changed according to

$${w}_{i , j}^{′ ( n )} = {w}_{i , j}^{( n )} - γ \cdot {δ}_{j}^{( n )} \cdot {x}_{i}^{( n )}$$

wherein γ is a learning rate, and the numbers

$${δ}_{j}^{( n )}$$

may b e recursively calculated as

$${δ}_{j}^{( n )} = ( {\sum }_{k} {δ}_{k}^{( n + 1 )} \cdot {w}_{j , k}^{( n + 1 )} ) \cdot {f}^{′} ( {\sum }_{i} {x}_{i}^{( n )} \cdot {w}_{i , j}^{( n )} )$$

based on

$${δ}_{j}^{( n + 1 )} ,$$

if the (n+1)-th layer is not the output layer, and based on

$${δ}_{j}^{( n )} = ( {x}_{k}^{( n + 1 )} - {t}_{j}^{( n + 1 )} ) \cdot {f}^{′} ( {\sum }_{i} {x}_{i}^{( n )} \cdot {w}_{i , j}^{( n )} )$$

if the (n+1)-th layer is the output layer 114, wherein f′ is the first derivative of the activation function, and

$${y}_{j}^{( n + 1 )}$$

is the comparison training value for the j-th node of the output layer 114.

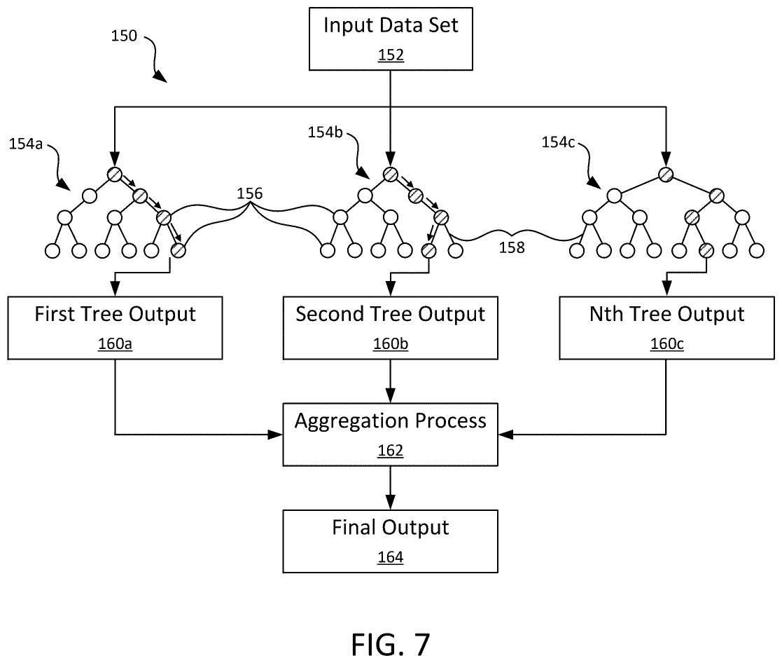

FIG. 7 illustrates a tree-based neural network 150, in accordance with some embodiments. In particular, the tree-based neural network 150 is a random forest neural network, though it will be appreciated that the discussion herein is applicable to other decision tree neural networks. The tree-based neural network 150 includes a plurality of trained decision trees 154a-154c each including a set of nodes 156 (also referred to as “leaves”) and a set of edges 158 (also referred to as “branches”).

Each of the trained decision trees 154a-154c may include a classification and/or a regression tree (CART). Classification trees include a tree model in which a target variable may take a discrete set of values, e.g., may be classified as one of a set of values. In classification trees, each leaf 156 represents class labels and each of the branches 158 represents conjunctions of features that connect the class labels. Regression trees include a tree model in which the target variable may take continuous values (e.g., a real number value).

In operation, an input data set 152 including one or more features or attributes is received. A subset of the input data set 152 is provided to each of the trained decision trees 154a-154c. The subset may include a portion of and/or all of the features or attributes included in the input data set 152. Each of the trained decision trees 154a-154c is trained to receive the subset of the input data set 152 and generate a tree output value 160a-160c, such as a classification or regression output. The individual tree output value 160a-160c is determined by traversing the trained decision trees 154a-154c to arrive at a final leaf (or node) 156.

In some embodiments, the tree-based neural network 150 applies an aggregation process 162 to combine the output of each of the trained decision trees 154a-154c into a final output 164. For example, in embodiments including classification trees, the tree-based neural network 150 may apply a majority-voting process to identify a classification selected by the majority of the trained decision trees 154a-154c. As another example, in embodiments including regression trees, the tree-based neural network 150 may apply an average, mean, and/or other mathematical process to generate a composite output of the trained decision trees. The final output 164 is provided as an output of the tree-based neural network 150.

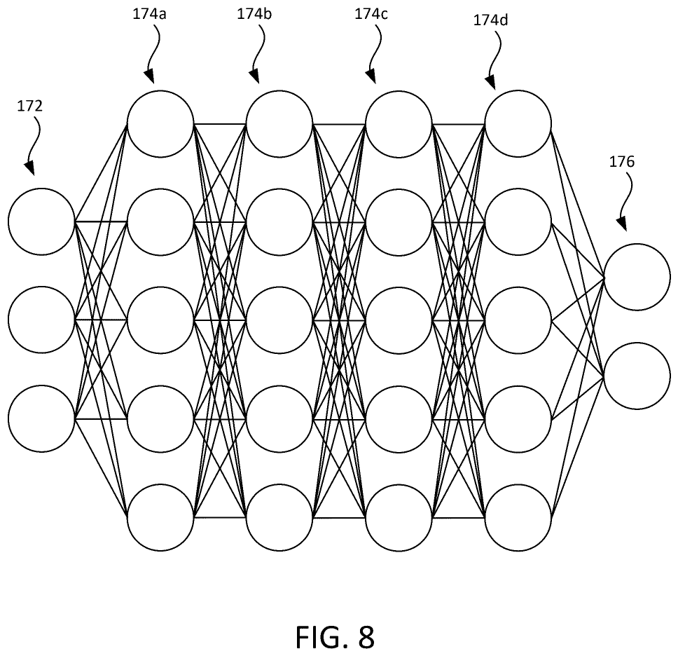

FIG. 8 illustrates a deep neural network (DNN) 170, in accordance with some embodiments. The DNN 170 is an artificial neural network, such as the neural network 100 illustrated in conjunction with FIG. 3, that includes representation learning. The DNN 170 may include an unbounded number of (e.g., two or more) intermediate layers 174a-174d each of a bounded size (e.g., having a predetermined number of nodes), providing for practical application and optimized implementation of a universal classifier. Each of the layers 174a-174d may be heterogenous. The DNN 170 may be configured to model complex, non-linear relationships. Intermediate layers, such as intermediate layer 174c, may provide compositions of features from lower layers, such as layers 174a, 174b, providing for modeling of complex data.

In some embodiments, the DNN 170 may be considered a stacked neural network including multiple layers each configured to execute one or more computations. The computation for a network with L hidden layers may be denoted as:

$$f ( x ) = f [ {a}^{( L + 1 )} ( {h}^{( L )} ( {a}^{( L )} ( … ( {h}^{( 2 )} ( {a}^{( 2 )} ( {h}^{( 1 )} ( {a}^{( 1 )} ( x ) ) ) ) ) ) ) ) ]$$

where a^{(l)}(x) is a preactivation function and h^{(l)}(x) is a hidden-layer activation function providing the output of each hidden layer. The preactivation function a^{(l)}(x) may include a linear operation with matrix W^{(l)} and bias b^{(l)}, where:

$${a}^{( l )} ( x ) = {W}^{( l )} x + {b}^{( l )}$$

In some embodiments, the DNN 170 is a feedforward network in which data flows from an input layer 172 to an output layer 176 without looping back through any layers. In some embodiments, the DNN 170 may include a backpropagation network in which the output of at least one hidden layer is provided, e.g., propagated, to a prior hidden layer. The DNN 170 may include any suitable neural network, such as a self-organizing neural network, a recurrent neural network, a convolutional neural network, a modular neural network, and/or any other suitable neural network.

In some embodiments, a DNN 170 may include a neural additive model (NAM). An NAM includes a linear combination of networks, each of which attends to (e.g., provides a calculation regarding) a single input feature. For example, a NAM may be represented as:

$$y = β + {f}_{1} ( {x}_{1} ) + {f}_{2} ( {x}_{2} ) + … + {f}_{K} ( {x}_{K} )$$

where β is an offset and each fᵢ is parametrized by a neural network. In some embodiments, the DNN 170 may include a neural multiplicative model (NMM), including a multiplicative form for the NAM mode using a log transformation of the dependent variable y and the independent variable x:

$$y = {e}^{β} {e}^{f ( log x )} {e}^{{\sum }_{i} {f}_{i}^{d} ( {d}_{i} )}$$

where d represents one or more features of the independent variable x.

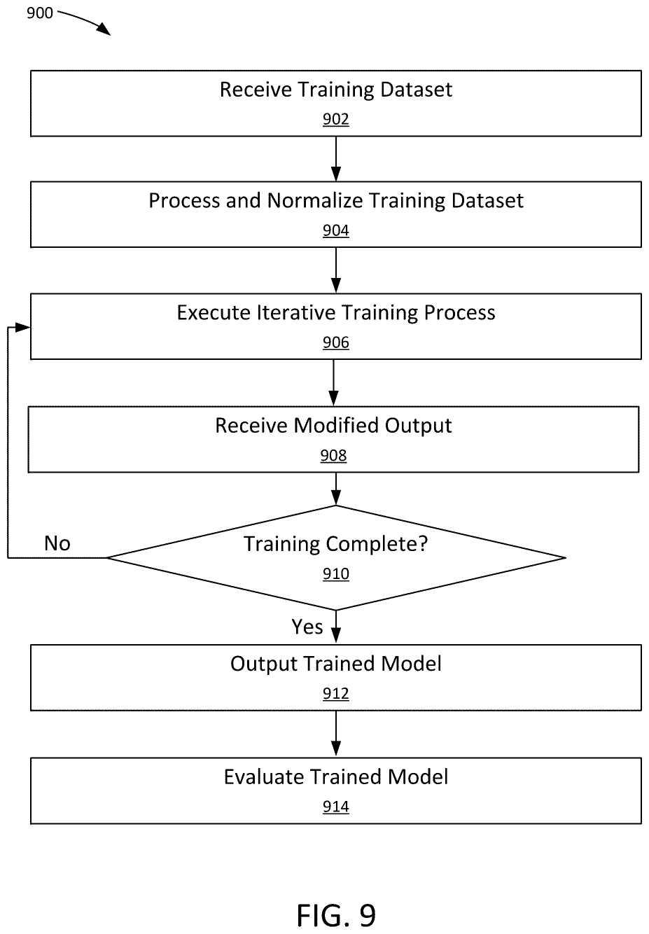

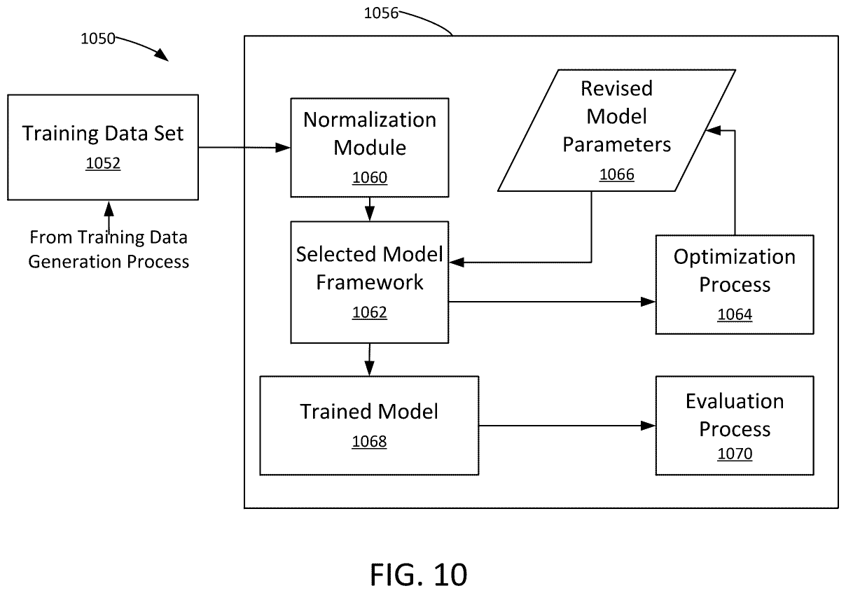

In some embodiments, a care management stratification engine (e.g., care management stratification engine 4002 in FIG. 4) and an alert generation engine (e.g., alert generation engine 5032 in FIG. 5) can include and/or implement one or more trained models, such as cost prediction model, illness severity prediction model, quality of service prediction model, and intervention events prediction model (e.g., as described with reference to FIG. 3). In some embodiments, one or more trained models can be generated using an iterative training process based on a training dataset. FIG. 9 illustrates a method 900 for generating one or more trained models, such as a trained cost prediction model, illness severity prediction model, quality of service prediction model, and intervention events prediction model (e.g., as described with reference to FIG. 3), in accordance with some embodiments. FIG. 10 is a process flow 950 illustrating various steps of the method 900 of generating a trained model, in accordance with some embodiments. At step 902, a training dataset 1052 is received by a system, such as a processing device 10. The training dataset 1052 can include labeled and/or unlabeled data.

At optional step 904, the received training dataset 1052 is processed and/or normalized by a normalization module 1060. In some embodiments, processing of the received training dataset 1052 includes outlier detection configured to remove data likely to skew training. In some embodiments, processing of the received training dataset 1052 includes removing features that have limited value with respect to training of the cost prediction model, illness severity prediction model, quality of service prediction model, and/or intervention events prediction model.

At step 906, an iterative training process is executed to train a selected model framework 1062. The selected model framework 1062 can include an untrained (e.g., base) machine learning model, such as cost prediction model, illness severity prediction model, quality of service prediction model, and intervention events prediction model and/or a partially or previously trained model (e.g., a prior version of a trained model). The training process is configured to iteratively adjust parameters (e.g., hyperparameters) of the selected model framework 1062 to minimize a cost value (e.g., an output of a cost function) for the selected model framework 1062.

The training process is an iterative process that generates set of revised model parameters 1066 during each iteration. The set of revised model parameters 1066 can be generated by applying an optimization process 1064 to the cost function of the selected model framework 1062. The optimization process 1064 can be configured to reduce the cost value (e.g., reduce the output of the cost function) at each step by adjusting one or more parameters during each iteration of the training process.

After each iteration of the training process, at step D08, a determination is made whether the training process is complete. The determination at step D08 can be based on any suitable parameters. For example, in some embodiments, a training process can complete after a predetermined number of iterations. As another example, in some embodiments, a training process can complete when it is determined that the cost function of the selected model framework D62 has reached a minimum, such as a local minimum and/or a global minimum.

At step 910, a trained model 1068, such as a trained cost prediction model, illness severity prediction model, quality of service prediction model, and intervention events prediction model is output and provided for use in a method for generating automated care management intervention, such as the for generating automated care management intervention 200 discussed above with respect to FIGS. 3-6. At optional step 912, a trained model 1068 can be evaluated by an evaluation process 1070. A trained model can be evaluated based on any suitable metrics, such as, for example, an F or F1 score, normalized discounted cumulative gain (NDCG) of the model, mean reciprocal rank (MRR), mean average precision (MAP) score of the model, and/or any other suitable evaluation metrics. Although specific embodiments are discussed herein, it will be appreciated that any suitable set of evaluation metrics can be used to evaluate a trained model.

Although the subject matter has been described in terms of exemplary embodiments, it is not limited thereto. Rather, the appended claims should be construed broadly, to include other variants and embodiments, which may be made by those skilled in the art.

What is claimed is:

1. A system, comprising:
a non-transitory memory;
a processor communicatively coupled to the non-transitory memory, wherein the processor is configured to read a set of instructions to:
generate a first risk score for each member in a population using a first predictive model, wherein the first predictive model is trained using historical data that includes medical cost and utilization of care management services;
generate a second risk score for each member in the population using a second predictive model different from the first predictive model, wherein the second predictive model is trained using historical data that includes medical conditions, pharmacy use, and lab results;
stratify the population into a plurality of risk groups based on: (i) predefined manageable medical conditions; (ii) the first risk score; and (iii) the second risk score;
based on the plurality of risk groups, segment the population into a high-risk group and a low-risk group, including assigning members of a first subset of the plurality of risk groups to the high-risk group and assigning members of a second subset of the plurality of risk group to the low-risk group; and
assign a panel of care management associates of a plurality of care management associates to each member in the member population based on whether the member belongs to the high-risk group or the low-risk group.

2. The system of claim 1, wherein the processor is configured to read a set of instructions to:
identify intervention events using a predictive model; and
automatically generate alerts based on the identified events for provision to respective panels of one or more care management associates.

3. The system of claim 1, wherein members of a respective risk group of the plurality of risk groups are assigned priority based on an engagement score determined by a third predictive model.

4. The system of claim 1, wherein a first predictive model includes extreme gradient boost framework.

5. The system of claim 1, wherein the second predictive model is a logistic regression model.

6. The system of claim 1, wherein the plurality of care management associates are segmented into a high-risk group and a low-risk group using a score generated by a fourth predictive model.

7. A computer-implemented method, comprising:
generating a first risk score for each member in a population using a first predictive model, wherein the first predictive model is trained using historical data that includes medical cost and utilization of care management services;
generating a second risk score for each member in the population using a second predictive model different from the first predictive model, wherein the second predictive model is trained using historical data that includes medical conditions, pharmacy use, and lab results; and
stratifying the population into a plurality of risk groups based on: (i) predefined manageable medical conditions; (ii) the first risk score; and (iii) the second risk score;
based on the plurality of risk groups, segmenting the population into a high-risk group and a low-risk group, including assigning members of a first subset of the plurality of risk groups to the high-risk group and assigning members of a second subset of the plurality of risk group to the low-risk group; and
assigning a panel of care management associates of a plurality of care management associates to each member in the member population based on whether the member belongs to the high-risk group or the low-risk group, wherein the plurality of care management associates are segmented into a high-risk group and a low-risk group.

8. The method of claim 7, including:
identifying intervention events using a predictive model; and
automatically generating alerts based on the identified events for provision to respective panels of one or more care management associates.

9. The method of claim 7, wherein members of a respective risk group of the plurality of risk groups are assigned priority based on an engagement score determined by a third predictive model.

10. The method of claim 7, wherein a first predictive model includes extreme gradient boost framework.

11. The method of claim 7, wherein the second predictive model is a logistic regression model.

12. The method of claim 7, wherein the plurality of care management associates are segmented into a high-risk group and a low-risk group using a score generated by a fourth predictive model.

13. A non-transitory computer readable medium having instructions stored thereon, wherein the instructions, when executed by at least one processor, cause at least one device to perform operations comprising:
generating a first risk score for each member in a population using a first predictive model, wherein the first predictive model is trained using historical data that includes medical cost and utilization of care management services;
generating a second risk score for each member in the population using a second predictive model different from the first predictive model, wherein the second predictive model is trained using historical data that includes medical conditions, pharmacy use, and lab results; and
stratifying the population into a plurality of risk groups based on: (i) predefined manageable medical conditions; (ii) the first risk score; and (iii) the second risk score;
based on the plurality of risk groups, segmenting the population into a high-risk group and a low-risk group, including assigning members of a first subset of the plurality of risk groups to the high-risk group and assigning members of a second subset of the plurality of risk group to the low-risk group; and
assigning a panel of care management associates of a plurality of care management associates to each member in the member population based on whether the member belongs to the high-risk group or the low-risk group, wherein the plurality of care management associates are segmented into a high-risk group and a low-risk group.

14. The non-transitory computer readable medium of claim 13, wherein the operations including:
identifying intervention events using a predictive model; and
automatically generating alerts based on the identified events for provision to respective panels of one or more care management associates.

15. The non-transitory computer readable medium of claim 13, wherein members of a respective risk group of the plurality of risk groups are assigned priority based on an engagement score determined by a third predictive model.

16. The non-transitory computer readable medium of claim 13, wherein a first predictive model includes extreme gradient boost framework.

17. The non-transitory computer readable medium of claim 13, wherein the second predictive model is a logistic regression model.

18. The non-transitory computer readable medium of claim 13, wherein the plurality of care management associates are segmented into a high-risk group and a low-risk group using a score generated by a fourth predictive model.
